(19) United States
(12) Patent Application Publication
(10) Pub. No.: US 2009/0233881 A1
(43) Pub. Date: Sep. 17, 2009

(54) COMPOUNDS HAVING ANTI-CANCER PROPERTIES

(75) Inventors: Simon Michael West, Victoria (AU); Esra Ogru, Victoria (AU); Robert Gianello, Victoria (AU)

Correspondence Address: MICHAEL BEST & FRIEDRICH LLP, 100 E WISCONSIN AVENUE, Suite 3300, MILWAUKEE, WI, 53202, US

(73) Assignee: Vital Health Sciences Pty. Ltd, Melbourne, Victoria (AU)

(21) Appl. No.: 11/817,439

(22) PCT Filed: Mar. 3, 2006
(86) PCT No.: PCT/AU2006/000280

§ 371 Date: Nov. 10, 2008

(30) Foreign Application Priority Data
Mar. 3, 2005 (AU) 2005 901014
Aug. 30, 2005 (AU) 2005 904735

(51) Int. Cl. A61K 31/66

(52) U.S. Cl. 514/75

(57) ABSTRACT

There is provided a method for alleviating symptoms, treating or preventing cancer, the method comprising administering to a subject, having or at risk of developing cancer, a pharmaceutical formulation comprising an effective amount of one or more phosphate derivatives of one or more hydroxy chromans selected from the group consisting of 7:8 dimethyl 6 hydroxy chromans, 8 methyl 6 hydroxy chromans and mixtures thereof.

### FIELD OF THE INVENTION

The present invention relates to compounds which induce cell apoptosis and may have anti-cancer properties.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Today, millions of people are living with cancer or have had cancer. Over one million people get cancer each year. Anyone can get cancer at any age; however, about 77% of all cancers are diagnosed in people aged 55 and older. The three most common cancers in men are prostate cancer, lung cancer, and colon cancer. In women, the three most frequently occurring cancers are breast cancer, lung cancer, and colon cancer.

Cancer develops when cells in a part of the body begin to grow out of control. Although there are many kinds of cancer, they all start because of out-of-control growth of abnormal cells. Normal body cells grow, divide, and die in an orderly fashion. During the early years of a person's life, normal cells divide more rapidly until the person becomes an adult. After that, cells in most parts of the body divide only to replace worn-out or dying cells and to repair injuries. Because cancer cells continue to grow and divide, they are different from normal cells. Instead of dying, they outlive normal cells and continue to form new abnormal cells. This growth can kill when these cells prevent normal function of vital organs or spread throughout the body, damaging essential systems. The sooner a cancer is found and treatment begins, the better are the chances for living for many years.

Cancer cells develop because of damage to DNA. Most of the time when DNA becomes damaged the body is able to repair it. In cancer cells, the damaged DNA is not repaired. People can inherit damaged DNA, which accounts for inherited cancers. Many times though, a person's DNA becomes damaged by exposure to something in the environment, like smoking. The risk of developing most types of cancer can be reduced by changes in a person's lifestyle, for example, by quitting smoking and eating according to a better diet.

Cancer cells often travel to other parts of the body where they begin to grow and replace normal tissue. This process, called metastasis, occurs as the cancer cells enter the bloodstream or lymph vessels of the body. Cells from a primary tumour which spread through the bloodstream may grow only in certain, and not all, organs.

There are at least 200 different kinds of cancers. They can develop in almost any organ, fluid or tissue. Different types of cancer can behave very differently. That is why people with cancer need treatment that is aimed at their particular kind of cancer.

The four major types of treatment for cancer are surgery, radiation, chemotherapy, and biologic therapies. There are also hormone therapies such as tamoxifen and transplant options such as those done with bone marrow.

Treatment varies based on the type of cancer and its stage. The stage of a cancer refers to how much it has grown and whether the tumour has spread from its original location. If the cancer is confined to one location and has not spread, the goal for treatment would be surgery and cure. If all of the cancer cannot be removed with surgery, the options for treatment include radiation, chemotherapy, or both. Some cancers require a combination of surgery, radiation, and chemotherapy.

While surgery and radiation therapy are used to treat localized cancers, chemotherapy is used to treat cancer cells that have metastasized (spread) to other parts of the body. Depending on the type of cancer and its stage of development, chemotherapy can be used to cure cancer, to keep the cancer from spreading, to slow the cancer's growth, to kill cancer cells that may have spread to other parts of the body, or to relieve symptoms caused by cancer.

The side effects of chemotherapy depend on the type of drugs, the amounts taken, and the length of treatment. The most common are nausea and vomiting, temporary hair loss, increased chance of infections, and fatigue. Many of these side effects can be uncomfortable or emotionally upsetting. However, most side effects can be controlled with medicines, supportive care measures, or by changing the treatment schedule.

There is still a need for chemotherapeutic drugs which have fewer side effects and which can be used to treat cancer lines which become resistant to current treatments.

### Lycopene

Lycopene, an open-chain unsaturated carotenoid without provitamin-A activity, is present in many fruits and vegetables. It is a red, fat-soluble pigment that imparts red colour to tomatoes, guava, rosehip, watermelon and pink grapefruit. Lycopene is a proven antioxidant. In the body, lycopene is deposited in the liver, lungs, prostate gland, colon and skin. Its concentration in body tissues tends to be higher than all other carotenoids (it accounts for 50% of all carotenoids in human serum).

Research shows that lycopene in tomatoes can be absorbed more efficiently by the body if processed into juice, sauce, paste and ketchup. The chemical form of lycopene found in tomatoes is converted by the temperature changes involved in processing to make it more easily absorbed by the body.

Tomatoes are the fourth most commonly consumed fresh vegetable and the most frequently consumed canned vegetable in the American diet. There is emerging epidemiology data supporting the connection between increased tomato consumption and reduced risk for both cardiovascular disease and prostate cancer. Ongoing preliminary research suggests that lycopene is associated with reduced risk of macular degenerative disease, serum lipid oxidation and cancers of the lung, bladder, cervix, skin, digestive tract, breast and prostate cancer. Studies are underway to investigate other potential benefits of lycopene.

### Tocopheryl Phosphate

Vitamin E is thought to have many beneficial properties which promote health including antioxidant properties. Vitamin E is considered to comprise 8 different forms: alpha, beta, delta and gamma tocopherols and alpha, beta, delta and gamma tocotrienols. Tocopherols differ from tocotrienols in that they have a saturated phytyl side chain rather than an unsaturated isoprenyl side chain. The four forms differ in the number of methyl groups on the chromanol group (alpha has three, beta and gamma have two and delta has one).

In international patent application no WO 03/026673, there is disclosure that having increased storage levels of vitamins, including tocopheryl phosphate, could be beneficial in alleviating or treating cancer where tocopherol affects cell adhesion. However, there is no disclosure of tocopheryl phosphate causing cell death or the difference in activity between alpha tocopherol and delta and gamma tocopherol.

Tocopheryl phosphate has also been disclosed in international patent application no WO 2004/064831 as having properties related to inhibiting the proliferation of monocytes/macrophages, proliferation of smooth muscle cells, the expression of CD36 receptors and the uptake of oxidized LDL. The examples show only an inhibition of cell growth and there is no disclosure of cell death. Further, there is no disclosure of treating cancer or the difference in activity between alpha tocopherol and delta and gamma tocopherol.

International patent application nos. WO 00/16772 and WO 03/039461 teach that naturally occurring alpha, gamma and delta tocotrienols as well as gamma and delta tocopherols exhibit anticancer activity. However, alpha tocopherol does not have anticancer properties. Further, these applications disclose that the use of perphosphate derivatives of tocopherol type compounds are useful for treating cancer. Human trials and surveys that have tried to associate free tocopherol intake with cancer incidence have been generally inconclusive and free tocopherols are not a useful clinical option for the treatment of cancer.

There is still a need for improved treatments for cancer.

### SUMMARY OF THE INVENTION

It has now surprisingly been found that phosphate derivatives of 7:8 dimethyl 6 hydroxy chromans and 8 methyl 6 hydroxy chromans (5 and 7 hydroxy chromans) are able to cause cell apoptosis and thus could be useful in the treatment of cancer, whereas the 5:7:8 tri-methyl 6 hydroxy chromans (a: hydroxy chromans) do not have this property.

It has also been shown that the combination of one or more anticancer agents and phosphate derivatives of 7:8 dimethyl 6 hydroxy chromans and 8 methyl 6 hydroxy chromans (δ and γ hydroxy chromans) can be effective to kill cancer cells using lower concentrations of the anti-cancer agent.

According to a first aspect of the invention, there is provided a method for alleviating symptoms, treating or preventing cancer, the method comprising administering to a subject, having or at risk of developing cancer, a pharmaceutical formulation comprising an effective amount of one or more phosphate derivatives of one or more hydroxy chromans selected from the group consisting of 7:8 dimethyl 6 hydroxy chromans, 8 methyl 6 hydroxy chromans and mixtures thereof.

According to a second aspect of the invention, there is provided a method for inducing cell apoptosis comprising administering to cells an effective amount of one or more phosphate derivatives of one or more hydroxy chromans selected from the group consisting of 7:8 dimethyl 6 hydroxy chromans, 8 methyl 6 hydroxy chromans and mixtures thereof.

According to a third aspect of the invention, there is provided a method for alleviating symptoms, treating or preventing cancer, the method comprising administering to a subject, having or at risk of developing cancer, an effective amount of a pharmaceutical formulation comprising:
(a) one or more anticancer agents; and
(b) one or more phosphate derivatives of one or more hydroxy chromans selected from the group consisting of 7:8 dimethyl 6 hydroxy chromans, 8 methyl 6 hydroxy chromans and mixtures thereof.

According to a fourth aspect of the invention, there is provided a method for inducing cell apoptosis comprising administering to cells an effective amount of a formulation comprising one or more anticancer agents and one or more phosphate derivatives of one or more hydroxy chromans selected from the group consisting of 7:8 dimethyl 6 hydroxy chromans, 8 methyl 6 hydroxy chromans and mixtures thereof.

According to a fifth aspect of the invention, there is provided a method for increasing the efficacy of lycopene, the method comprising combining lycopene with one or more phosphate derivatives of one or more hydroxy chromans selected from the group consisting of 7:8 dimethyl 6 hydroxy chromans, 8 methyl 6 hydroxy chromans and mixtures thereof.

This aspect of the invention includes a pharmaceutical formulation comprising an effective amount of lycopene and an effective amount of one or more phosphate derivatives of one or more hydroxy chromans selected from the group consisting of 7:8 dimethyl 6 hydroxy chromans, 8 methyl 6 hydroxy chromans and mixtures thereof.

In a further aspect, the invention provides a method for increasing the efficacy of an anticancer agent, the method comprising combining the anticancer agent with one or more phosphate derivatives of one or more hydroxy chromans selected from the group consisting of 7:8 dimethyl 6 hydroxy chromans, 8 methyl 6 hydroxy chromans and mixtures thereof. An examples of an appropriate anticancer agent is tamoxifen.

In a further aspect, the invention provides a pharmaceutical formulation when used for inducing cell apoptosis, the formulation comprising one or more anticancer agents and one or more phosphate derivatives of one or more hydroxy chromans selected from the group consisting of 7:8 dimethyl 6 hydroxy chromans, 8 methyl 6 hydroxy chromans and mixtures thereof.

In a further aspect, the invention provides a pharmaceutical formulation when used for alleviating symptoms, treating or preventing cancer, the formulation comprising one or more anticancer agents and one or more phosphate derivatives of one or more hydroxy chromans selected from the group consisting of 7:8 dimethyl 6 hydroxy chromans, 8 methyl 6 hydroxy chromans and mixtures thereof.

In a further aspect, the invention provides for use of one or more anticancer agents and one or more phosphate derivatives of one or more hydroxy chromans selected from the group consisting of 7:8 dimethyl 6 hydroxy chromans, 8 methyl 6 hydroxy chromans and mixtures thereof, together with a suitable carrier or diluent in the manufacture of a medicament for alleviating symptoms, treating or preventing cancer.

In a further aspect, the invention provides a pharmaceutical composition when used for inducing cell apoptosis, the composition comprising an effective amount of one or more phosphate derivatives of one or more hydroxy chromans selected from the group consisting of 7:8 dimethyl 6 hydroxy chromans, 8 methyl 6 hydroxy chromans and mixtures thereof.

In a further aspect, the invention provides for use of an effective amount of one or more phosphate derivatives of one or more hydroxy chromans selected from the group consisting of 7:8 dimethyl 6 hydroxy chromans, 8 methyl 6 hydroxy chromans and mixtures thereof, together with a suitable carrier or diluent in the manufacture of a medicament for alleviating symptoms, treating or preventing cancer.

Anti-cancer treatments often include the use of a cocktail of cytotoxic reagents. The dose form may contain other pharmaceutical compounds which do not antagonise the activity of the phosphate derivatives of hydroxy chromans. The other pharmaceutical compound may be administered before, with or after the one or more phosphate derivatives of one or more hydroxy chromans. Examples of suitable other pharmaceutical compounds include taxol, docetaxel, adriamycin, tamoxifen and doxorubicin.

The term “effective amount” is used herein to refer to an amount which is sufficient to induce cell apoptosis or for alleviating symptoms, treating or preventing cancer.

A person skilled in the art will know which anticancer agents are suitable for use in the invention. The term “anticancer agents” is used herein to include, but is not limited to, all pro-apoptotic compounds as well as alkylating agents, antimetabolite agents, immunological agents, compounds that influence signal transduction pathways and other chemotherapeutic agents. Preferably, the one or more anticancer agents is lycopene or tamoxifen.

The term “hydroxy chromans” is used herein to refer to the hydroxy derivatives of chromans. The hydroxy chroman derivatives relevant to this invention are the 7:8 dimethyl 6 hydroxy chromans and 8 methyl 6 hydroxy chromans isomers whether in enantiomeric or raecemic forms. More preferably, the hydroxy chroman is selected from the group consisting of the δ and γ tocols and mixtures thereof. The tocols include the δ and γ isomers of derivatives of 6:hydroxy 2:methyl chroman (see structure below) where R₁, R₂ and R₃ may be hydrogen or methyl groups, that is, the γ-7:8 di-methyl and δ-8 methyl derivatives. In the tocopherols, R₄ is substituted by 4:8:12 trimethyl tridecyl and the 2, 4, and 8 positions (see *) may be stereoisomer's with R or S activity or racemic. In the tocotrienols, R₄ is substituted by 4:8:12 trimethyl trideca-3:7:11 triene and the 2 position may be sterioactive as R or S stereoisomers or racemic.

The term “phosphate derivatives” is used herein to refer to the acid forms of phosphorylated electron transfer agents, salts of the phosphates including metal salts such as sodium, magnesium, potassium and calcium and any other derivative where the phosphate proton is replaced by other substituents such as ethyl or methyl groups or phosphatidyl groups. However, the term does not include perphosphates. The term includes mixtures of phosphate derivatives, especially those which result from phosphorylation reactions, as well as each of the phosphate derivatives alone. For example, the term includes a mixture of mono-tocopheryl phosphate (TP) and di-tocopheryl phosphate (T2P) as well as each of TP and T2P alone. Suitable mixtures are described in international patent application no PCT/AU01/01475.

Preferably, the one or more phosphate derivatives of one or more electron transfer agents is selected from the group consisting of mono-tocopheryl phosphate, di-tocopheryl phosphate, mono-tocotrienyl phosphate, di-tocotrienyl phosphate and mixtures thereof. Most preferably, the one or more phosphate derivatives of one or more electron transfer agents is a mixture of one or more of mono-tocopheryl phosphate, di-tocopheryl phosphate, mono-tocotrienyl phosphate and di-tocotrienyl phosphate.

In some situations, it may be necessary to use a phosphate derivative such as a phosphatide where additional properties such as increased water solubility are preferred. Phosphatidyl derivatives are amino alkyl derivatives of organic phosphates. These derivatives may be prepared from amines having a structure of R₁R₂N(CH₂)ₙOH wherein n is an integer between 1 and 6 and R₁ and R₂ may be either H or short alkyl chains with 3 or less carbons. R₁ and R₂ may be the same or different. The phosphatidyl derivatives are prepared by displacing the hydroxyl proton of the electron transfer agent with a phosphate entity that is then reacted with an amine, such as ethanolamine or N,N′ dimethylethanolamine, to generate the phosphatidyl derivative of the electron transfer agent. One method of preparation of the phosphatidyl derivatives uses a basic solvent such as pyridine or triethylamine with phosphorous oxychloride to prepare the intermediate which is then reacted with the hydroxy group of the amine to produce the corresponding phosphatidyl derivative, such as P cholyl P tocopheryl dihydrogen phosphate.

In some situations, complexes of phosphate derivatives of the electron transfer agents may also be utilized where additional properties such as improved stability or deliverability may be useful. The term “complexes of phosphate derivatives” refers to the reaction product of one or more phosphate derivatives of electron transfer agents with one or more complexing agents selected from the group consisting of amphoteric surfactants, cationic surfactants, amino acids having nitrogen functional groups and proteins rich in these amino acids as disclosed in international patent application no PCT/AU01/01476, incorporated herein by reference. Examples of proteins rich in these amino acids are those proteins having either at least 1 in 62 amino acids as arginine, or at least 1 in 83 histidine, or at least 1 in 65 as lysine, such as the various forms of the protein casein. Other examples include insulin, parathyroid hormone (PTH), glucagon, calcitonin, adrenocorticotropic hormone (ACTH), prolactin, interferon-α and -β and -γ, leutenising hormone (LH) (also known as gonadotropin releasing hormone), follicle stimulating hormone (FSH) and colony stimulating factor (CSF). The amino acid composition of most of these examples is listed in the table.

| Amino acids in protein | Amino acids | Ratio of Total Amino acids |
|---|---|---|
| Insulin | 110 | |
| arg | 5 | 1 in 22 |
| his | 2 | 1 in 55 |
| lys | 2 | 1 in 55 |
| PTH | 84 | |
| arg | 5 | 1 in 17 |
| his | 0 | 0 |
| lys | 5 | 1 in 17 |
| Glucagon | 180 | |
| arg | 16 | 1 in 11 |
| his | 4 | 1 in 45 |
| lys | 10 | 1 in 18 |
| Calcitonin | 93 | |
| arg | 6 | 1 in 16 |
| his | 3 | 1 in 31 |
| lys | 5 | 1 in 19 |
| ACTH | 41 | |
| arg | 3 | 1 in 14 |
| his | 1 | 1 in 41 |
| lys | 4 | 1 in 10 |
| Prolactin | 220 | |
| arg | 12 | 1 in 18 |
| his | 9 | 1 in 13 |
| lys | 11 | 1 in 11 |
| Interferon - | 133 | |
| alpha and beta | | |
| arg | 7 | 1 in 19 |
| his | 2 | 1 in 83 |
| lys | 7 | 1 in 19 |
| Interferon -gamma | 166 | |
| arg | 8 | 1 in 21 |
| his | 2 | 1 in 83 |
| lys | 21 | 1 in 8 |
| LH | 92 | |
| arg | 5 | 1 in 18 |
| his | 2 | 1 in 46 |
| lys | 7 | 1 in 13 |
| FSH | 129 | |
| arg | 5 | 1 in 26 |
| his | 2 | 1 in 65 |
| lys | 9 | 1 in 14 |
| CSF | 144 | |
| arg | 6 | 1 in 24 |
| his | 3 | 1 in 48 |
| lys | 6 | 1 in 24 |
| GH domain AOD9604 | 16 | |
| arg | 2 | 1 in 8 |

The preferred complexing agents are selected from the group consisting of arginine, lysine and tertiary substituted amines, such as those according to the following formula:

NR¹R²R³

wherein R¹ is chosen from the group comprising straight or branched chain mixed alkyl radicals from C6 to C22 and carbonyl derivatives thereof;
R² and R³ are chosen independently from the group comprising H, CH₂COOX, CH₂CHOHCH₂SO₃X, CH₂CHOHCH₂OPO₃X, CH₂CH₂COOX, CH₂CH₂CHOHCH₂SO₃X or CH₂CH₂CHOHCH₂OPO₃X and X is H, Na, K or alkanolamine provided R² and R³ are not both H; and
wherein when R¹ is RCO then R² may be CH₃ and R³ may be (CH₂CH₂)N(C₂H₄₀H)—H₂CHOPO₃ or R² and R³ together may be N(CH₂)₂N(C₂H₄OH)CH₂COO—.

Preferred complexing agents include arginine, lysine or lauryliminodipropionic acid where complexation occurs between the alkaline nitrogen centre and the phosphoric acid ester to form a stable complex.

The phosphate derivative of the hydroxy chroman may be administered to humans or animals through a variety of dose forms such as supplements, enteral feeds, parenteral dose forms, suppositories, oral dose forms, aerosols, intraocular forms, pulmonary and nasal delivery forms, dermal delivery including patches and creams.

For example, the phosphate derivative of the hydroxy chroman may be administered by an orally or parenterally administered dose form. These include tablets, powders, chewable tablets, capsules, oral suspensions, suspensions, emulsions or fluids, children's formulations and enteral feeds.

The dose form may further include any additives routinely used in preparation of that dose form such as starch or polymeric binders, sweeteners, coloring agents, emulsifiers, coatings and the like. Other suitable additives will be readily apparent to those skilled in the art.

In one embodiment, the dose form has an enteric coating as disclosed in international patent application PCT/AU01/01206, incorporated herein by reference.

In another embodiment, the dose form is a topical formulation as disclosed in international patent application PCT/AU02/01003, incorporated herein by reference.

Preferably, the subject is an animal. More preferably, the animal is a mammal. Most preferably, the mammal is a human.

### DRAWINGS

Various embodiments/aspects of the invention will now be described with reference to the following drawings in which,

FIG. 1 shows the results from Example 1.

FIG. 2 shows the effects on a prostate cancer cell line (DU-145) from Example 2.

FIG. 3 shows the effects on MCF-7 breast cancer cell proliferation from Example 3.

FIG. 4 shows the relative activity of different gamma tocopheryl phosphates from Example 4.

### EXAMPLES

Various embodiments/aspects of the invention will now be described with reference to the following non-limiting examples.

### Example 1

This study compared the efficacy or potency of the various forms of tocopherols (α, γ and δ) with their phosphorylated partners from ADM and BASF to inhibit Rat Aortic Smooth Muscle Cells (RASMC) proliferation.

### Materials

96 well plates (MTS viable cell assay)
6 well plates (Actual cell count assay)
DMEM/F12 Medium—GIBCO/Life Technologies
Phosphate buffered Saline (PBS)
Fetal Bovine Serum (FBS)
Rat Aortic Smooth Muscle Cells (RASMCs) p: 6-8 Cell Applications, Inc.
Cell Titer 96 Aqueous One Solution (MTS)—Promega
Trypsin/EDTA Solution (R-001-100)—Chemicon
Trypsin neutralizing solution (R-002-100)—Chemicon
Ethanol
Hemocytometer
Trypan blue (0.5% w/v in PBS)
Tocopheryl phosphate mixtures (mono-tocopheryl phosphate and di-tocopheryl phosphate) of the α, γ and δ isomers

### Methods

Rat Aortic Smooth Muscle Cell Proliferation—MTS Assays: The effect of α, δ and γ tocopherols and their phosphorylated counterparts was assessed in RASMC. A total of 3 concentrations were tested for each compound: 100, 500 and 1,000 μg/ml. The Rat Aortic Smooth Muscle Cells (RASMC) were seeded in growth medium (DMEM/F12+10% FBS) into 96 well plates (5,000 cells/well) maintained at 37° C., 5% CO₂). After 24 h, the growth media was removed and replaced with Basal DMEM/F12 media. Cells were serum starved for 48 hours to synchronize the cells. The basal media was then replaced by growth media plus the various treatments, for a further 4 days. Treatments were then prepared as stock solutions in either 100% ethanol (for alpha-T, alpha-TP, gamma-T and delta-T) or 100% acetic acid (for gamma-TP and delta-TP) and then diluted appropriately for the final cell concentration such that the final ethanol concentration did not exceed 0.1% and the final acetic acid concentration did not exceed 0.02%. Under these assay conditions these vehicle concentrations did not significantly alter RASMC proliferation. Each treatment was conducted with 8 replicates. At the end of the treatment period, 20 μl MTS reagent was added to each well and the absorbance at 490 nm was read after a further 1 hour incubation at 37° C., 5% CO₂. The CellTiter 96® Aqueous proliferation assay is a colorimetric method for determining the number of viable cells in proliferation assays. The CellTiter 96® Aqueous is composed of solutions of a novel tetrazolium compound (3-(4,5-dimethylthiazol-2-yl)-5-(3-carboxymethoxyphenyl)-2-(4-sulfophenyl)-2H-tetrazolium, inner salt; MTS) and an electron coupling reagent (phenazine methosulphate; PMS). MTS is bioreduced by cells into a formazan product that is soluble in tissue culture medium. The absorbance of the formazan at 490 nm can be measured directly from the 96-well plates and the absorbance is directly proportional to cell number (i.e. the greater the absorbance the greater the number of viable cells).

### Results and Conclusion

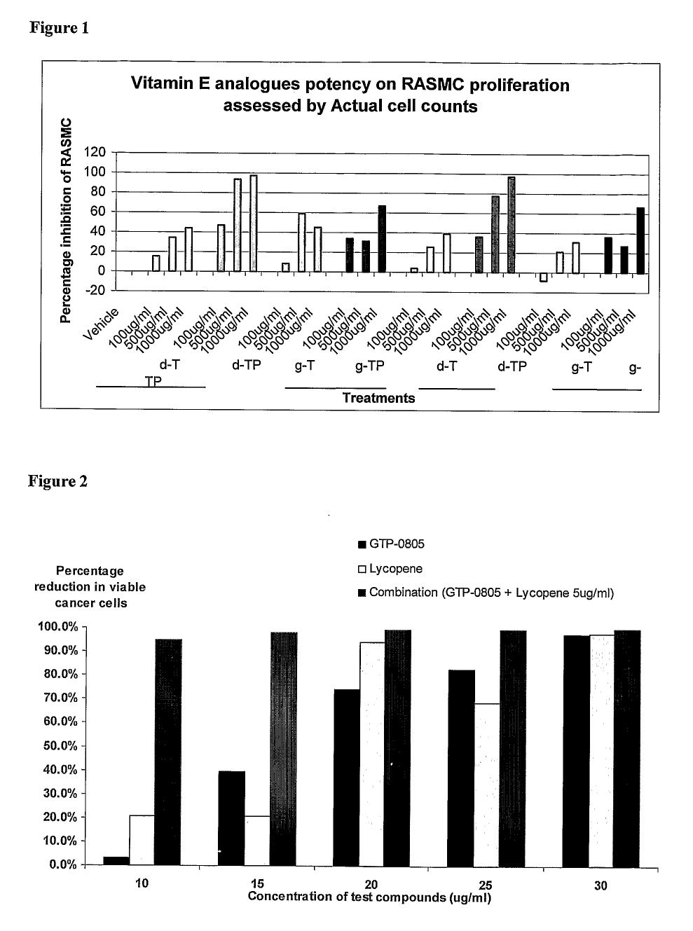

FIG. 1 shows the percentage inhibition of RASMC proliferation assessed by actual cell counts, on δ- and γ-tocopherols and their phosphorylated counterparts. The results demonstrate that γ and δ tocopheryl phosphate mixtures induced apoptosis (cell death) in the RASMC model (only 10% of cells incorporated the dye suggesting that 90% of cells had undergone apoptosis). Further, the results show that the γ and δ tocopheryl phosphate mixtures induce significant apoptosis whereas the nonphosphorylated form does not. The δ-tocopheryl phosphate mixtures from both ADM and BASF had the greatest efficacy compared to the other analogues tested. The effects also appear to be dose-dependent.

This is also very different to the effect of α-tocopheryl phosphate which does not induce apoptosis in the RASMC, it simply prevents excessive cellular proliferation through a regulated mechanism. With α-tocopheryl phosphate, RASMCs did not multiply and all cells were healthy and viable (as detected through the uptake of the dye). Whereas in the case of γ and δ tocopheryl phosphate, the RASMCs did not multiply and the remaining cells were not viable. This indicates a different mechanism of action.

### Example 2

This study compared the effect of lycopene and γ tocopheryl phosphate mixture, both individually and together, on prostate cancer cells.

### Materials and Methods

Culture of stock cells. DU-145 prostate cancer cells were purchased from American Type Culture Collection (Manassas, Va., USA). Stock cells were grown in Dulbecco's Modified Eagle Medium (DMEM) (Gibco BRL, Grand Island N.Y.) supplemented with 5% FBS (Fetal Bovine Serum, Gibco BRL, Grand Island N.Y.) in a humidified atmosphere of 5% CO₂ in air at 37° C. Cells were subcultured every 1-2 times a week.

Cell growth assay. Cells were trypsinized from the stock plates by treatment with trypsin/versene, added to an equal volume of phenol red-free RPMI-1640 (Gibco BRL, Grand Island N.Y.) supplemented with 5% dextran-charcoal treated fetal calf serum (DCFCS). Cells were resuspended to a cell count of 0.1×10⁵ cells/ml with the use of a haemocytometer and plated in monolayer in 0.5 ml aliquots into 24-well plastic culture dishes (Costar, Corning USA). After 24 hours, cells were treated with appropriate concentrations (see table) of γ-tocopheryl phosphate mixture (γ-TP) (Vital Health) and Lycopene (Sigma) or combinations of Lycopene and γ-TP diluted in phenol red-free RPMI medium 1640 supplemented with 5% DCFCS. The culture medium was changed every 3-4 days. The combination treatment contained lycopene and γ-TP in a 1:1 ratio by molecular weight/mass with lycopene varying from 5 ug/ml-40 ug/ml.

Cell counting. The cells were washed twice with 0.9% NaCl to remove non-adherent dead cells and were then lysed in 0.5 ml 2.5 mM Hepes buffer/1.5M MgCl₂ plus two drops of zapoglobin II lytic reagent (Beckman Coulter, Coulter Corp USA) for 5-15 minutes. The nuclei released were suspended in isoton III (Beckman Coulter, Coulter Corp, USA) and counted on a Coulter counter with particle size set at >5 μm. All cell counts were carried out in triplicate on triplicate well contents. The results were calculated as the average ±standard error. P-values were determined using Independent samples T-Test (by standard software packages SPSS).

### Results

The results are set out in the following tables and corresponding figures

TABLE 1
Results from γ tocopheryl phosphate mixture at 12 days

| | Concentration Gamma-TP (ug/ml) | | | | | | |
|---|---|---|---|---|---|---|---|
| | 0 | 10 | 15 | 20 | 25 | 30 | 40 |
| Total viable cells/ | 5.617 | 5.103 | 3.400 | 1.603 | 0.859 | 0.113 | 0.007 |
| well (×10⁵) | 5.992 | 5.851 | 3.464 | 1.447 | 1.052 | 0.192 | 0.005 |
| | 5.901 | 5.713 | 3.530 | 1.419 | 1.074 | 0.168 | 0.008 |
| | 5.844 | | | | | | |
| | 5.835 | | | | | | |
| | 5.239 | | | | | | |
| Average (×10⁵) | 5.738 | 5.556 | 3.465 | 1.490 | 0.995 | 0.157 | 0.007 |
| Std. Dev. (×10⁵) | 0.274 | 0.398 | 0.065 | 0.099 | 0.118 | 0.040 | 0.001 |

TABLE 2
Results from lycopene at 12 days

| | Concentration Lycopene (ug/ml) | | | | | | |
|---|---|---|---|---|---|---|---|
| | 0 | 5 | 10 | 15 | 20 | 25 | 30 |
| Total viable cells/ | 4.677 | 4.392 | 3.555 | 3.704 | 0.127 | 1.759 | 0.212 |
| well (×10⁵) | 4.984 | 4.383 | 3.869 | 3.727 | 0.222 | 1.196 | 0.075 |
| | 4.922 | 4.325 | | | 0.478 | | 0.073 |
| | 4.724 | | | | | | |
| | 4.453 | | | | | | |
| | 4.317 | | | | | | |
| Average (×10⁵) | 4.680 | 4.367 | 3.712 | 3.716 | 0.276 | 1.478 | 0.120 |
| Std. Dev. (×10⁵) | 0.259 | 0.036 | 0.222 | 0.016 | 0.182 | 0.398 | 0.080 |

TABLE 3
Results from combined lycopene and γ tocopheryl
phosphate mixture at 8 days

| | Concentration Gamma-TP (ug/ml) | | | | | | |
|---|---|---|---|---|---|---|---|
| | 0 | 10 | 15 | 20 | 25 | 30 | 40 |
| Total viable cells/ | 1.348 | 0.071 | 0.040 | 0.007 | 0.010 | 0.006 | 0.005 |
| well (×10⁵) | 1.673 | 0.074 | 0.020 | 0.010 | 0.010 | 0.010 | 0.005 |
| | 1.110 | | | | 0.010 | | 0.000 |
| | 1.391 | | | | | | |
| Average (×10⁵) | 1.381 | 0.073 | 0.030 | 0.009 | 0.010 | 0.008 | 0.003 |
| Std. Dev. (×10⁵) | 0.231 | 0.002 | 0.014 | 0.002 | 0.000 | 0.003 | 0.003 |

FIG. 2 shows the results from the above three tables (effects of γ-TP mixture (GTP-0805), lycopene (2 μg/ml), and in combination, on a prostate cancer cell line (DU-145)) expressed as percentage reduction in viable cells.

### Conclusion

The results show that the combination of lycopene and γ tocopheryl phosphate mixture was effect to kill the prostate cancer cells within just 8 days. Further, the results show that more prostate cancer cells were killed with a much lower concentration of lycopene in the combined treatment than with lycopene alone. The results also show that γ tocopheryl phosphate mixture is a potent apoptotic agent.

### Example 3

The in vitro effects of γ-TP mixture alone and in combination with tamoxifen, a commonly used anti-cancer drug, were investigated in breast (MCF-7) cancer cell lines.

### Methodology

Culture of stock cells: MCF-7 human breast cancer cells were kindly provided by Dr. K. Osborne at passage number 390. Stock cells were grown as monolayer cultures in Dulbecco's Modified Eagle Medium (DMEM) (Gibco BRL, Grand Island N.Y.) supplemented with 5% FBS (Gibco BRL, Grand Island N.Y.), 10-8 M estradiol in a humidified atmosphere of 5% CO2 in air at 37° C. 17 β-estradiol (cell cycle activator) was dissolved in ethanol and diluted 1:10,000 in culture medium. Cells were subcultured at weekly intervals by suspension with 0.06% trypsin/0.02% EDTA (pH 7.3).

Cell growth assay: Cells were suspended from the stock plates by treatment with trypsin/versene, added to an equal volume of phenol red-free RPMI medium 1640 (Gibco BRL, Grand Island N.Y.) supplemented with 5% dextran-charcoal treated FCS (DCFCS). Cells were resuspended to a cell count of 0.1×10⁵ cells/ml with the use of a haemocytometer and plated in monolayer in 0.5 ml aliquots into 24-well plastic culture dishes (Costar, Corning USA). After 24 hours, cells were treated with appropriate concentrations of tamoxifen, lycopene, γ-TP mixture, γ-T (Vital Health), or combinations, with or without estradiol diluted in phenol red-free RPMI medium 1640 supplemented with 5% DCFCS. The culture medium was changed every 3-4 days.
Cell counting: The cells were washed twice with 0.9% NaCl to remove non-adherent dead cells and were then lysed in 0.5 ml 2.5 mM Hepes buffer/1.5M MgCl₂ plus two drops of zapoglobin II lytic reagent (Beckman Coulter, Coulter Corp USA) for 5-15 minutes. The nuclei released were suspended in isoton III (Beckman Coulter, Coulter Corp, USA) and counted on a Coulter counter with particle size set at >5 μm. All cell counts were carried out in triplicate on triplicate well contents. The results were calculated as the average ±standard error. P-values were determined using Independent samples T-Test (by standard software packages SPSS).

### Results

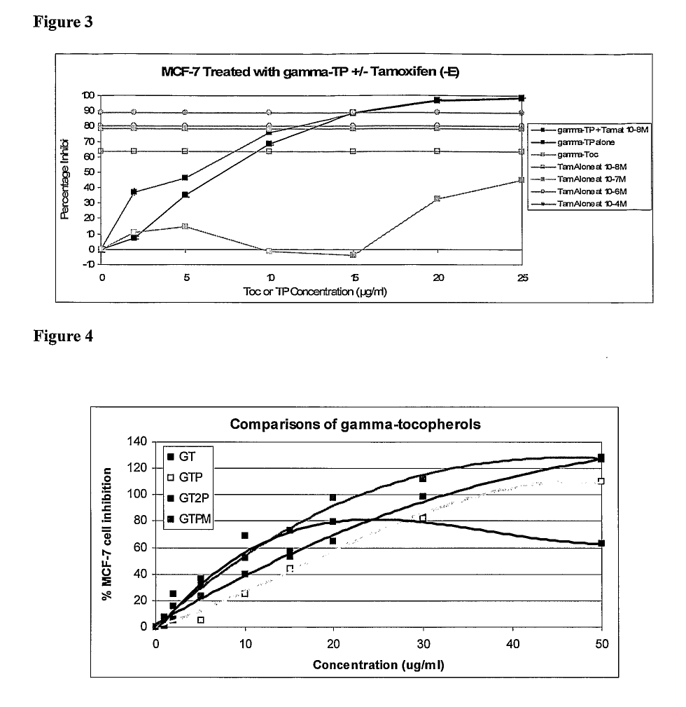

FIG. 3 shows the effects on MCF-7 breast cancer cell proliferation at varied doses of tamoxifen (Tam), γ-T (gamma-Toc), γ-TP (gamma-TP mixture) alone and γ-TP mixture plus tamoxifen (10⁻⁸M), without estradiol (−E). The combination of γ-TP mixture and the lowest dose of tamoxifen (10⁻⁸M) has a greater inhibitory effect than the highest dose of tamoxifen, suggesting a synergistic effect.

### Conclusion

In vitro results demonstrate that γ-TP mixture has potent anti-proliferative and pro-apoptotic activity when administered alone and in combination with agents such as tamoxifen. γ-TP mixture is very potent in breast cancer MCF-7 cell lines. At lower doses it is as potent as tamoxifen in the breast cancer cells. Synergistic effects can be seen with tamoxifen (at low doses). In addition, γ-TP mixture inhibits the growth of the cancer cells in a dose dependent manner.

### Example 4

In this example, the in vitro activity of gamma-tocopheryl phosphates (γ-T, γ-TP, γ-T2P and γ-TPM) in MCF-7 breast cancer cells was investigated.

MCF-7 breast cancer cell growth conditions: Cells were grown in 75 cm² plastic tissue cell flasks as monolayer in Dulbecco's Modified Eagle Medium (DMEM), supplemented with 10% FBS in a humidified atmosphere of 5% CO₂ in 95% air at 37° C. Cells were sub-cultured at bi-weekly intervals by suspension with 0.06% trypsin/0.02% EDTA (pH 7.3).

MCF-7 breast cancer cell line proliferation assays (MITS Assays): Cells were trypsinised (as performed during sub-culturing) in DMEM, supplemented with 10% FBS. Cells were re-suspended to a cell count of 10,000 cells/ml, with the use of a haemocytometer. Cells were seeded at 1,000 cells/well or by the addition of 100 μl of the cell suspension into 96-well cell culture plates. The cells were left overnight and then were synchronised (by serum starving for 24 hours), prior to the start of experiments.

After the cells were synchronised the cells were treated with the appropriate concentrations of the treatments, prepared in 100% ethanol (2, 5, 10, 15, 20, 30 & 50 μg/ml), they were added to RPMI medium 1640 supplemented with 10% dextran-charcoal treated FCS (DCFBS). The final ethanol concentration exposed to the cells did not exceed 1%. After 72 hours the plates are incubated with MTS reagent (as described in Example 1) for 1 hr. The plate was read in a spectrophotometer at 490 nm. There were 8 replicates for each compound tested (at the various concentrations shown below).

Treatment abbreviations: GT=gamma-tocopherol; GTP=gamma-tocopheryl phosphate; GT2P=gamma-di-tocopheryl phosphate, GTPM=gamma-tocopheryl phosphate mixture (combination of GTP and GT2P). Please note 0 μg/ml indicates that the vehicle control used (i.e. 1% ethanol).

Experiments carried out:
GT Alone (no E) at 0, 2, 5, 10, 15, 20, 30 & 50 μg/ml
GTP Alone (no E) at 0, 2, 5, 10, 15, 20, 30 & 50 μg/ml
GT2P Alone (no E) at 0, 2, 5, 10, 15, 20, 30 & 50 μg/ml
GTPM Alone (no E) at 0, 2, 5, 10, 15, 20, 30 & 50 μg/ml

### Results

The results are set out in the table below and in FIG. 4.

| | Concentration | | | | | | | | |
|---|---|---|---|---|---|---|---|---|---|
| Treatment | 0 | 1 | 2 | 5 | 10 | 15 | 20 | 30 | 50 |
| GT | 0 | −6.104 | 15.685 | 36.36 | 68.689 | 56.82 | 79.766 | 82.743 | 62.622 |
| GTP | 0 | 7.32 | 5.624 | 4.807 | 25.102 | 43.512 | 64.719 | 81.81 | 109.928 |
| GT2P | 0 | 7.283 | 4.91 | 31.07 | 39.471 | 53.126 | 64.557 | 98.43 | 126.506 |
| GTPM | 0 | 0.927 | 24.929 | 23.11 | 52.068 | 73.217 | 98.11 | 112.197 | 127.996 |

### Conclusion

The results show that GTPM was the most potent anti-cancer treatment, followed by GT2P, GTP, and GT was the least potent with limited activity. The findings show a significant reduction in cancer cell growth when cells are treated with the gamma tocopheryl phosphates, indicating that GTP,GT2P and GTPM may treat or slow the formation and progress of cancer.

The word ‘comprising’ and forms of the word ‘comprising’ as used in this description and in the claims does not limit the invention claimed to exclude any variants or additions. Modifications and improvements to the invention will be readily apparent to those skilled in the art. Such modifications and improvements are intended to be within the scope of this invention.

1. A method for alleviating symptoms, treating or preventing cancer, the method comprising administering to a subject, having or at risk of developing cancer, a pharmaceutical formulation comprising an effective amount of one or more phosphate derivatives of one or more hydroxy chromans selected from the group consisting of 7:8 dimethyl 6 hydroxy chromans (delta), 8 methyl 6 hydroxy chromans (gamma) and mixtures thereof.

2. The method according to claim 1 wherein the phosphate derivatives of one or more hydroxy chromans is selected from the group consisting of mono-tocopheryl phosphate, di-tocopheryl phosphate, mono-tocotrienyl phosphate, di-tocotrienyl phosphate and mixtures thereof.

3. The method according to claim 2 wherein the phosphate derivatives of hydroxy chromans is a mixture of mono-tocopheryl phosphate and di-tocopheryl phosphate.

4. The method according to claim 3 wherein the phosphate derivatives of hydroxy chromans is a mixture of mono-8 methyl 6 hydroxy tocopheryl phosphate (gamma) and di-8 methyl 6 hydroxy tocopheryl phosphate (gamma).

5. The method according to claim 1 further comprising the step of administering one or more other pharmaceutical compounds which do not antagonise the activity of the phosphate derivative of a hydroxy chroman.

6. The method according to claim 5 wherein the other pharmaceutical compounds are selected from the group comprising taxol, docetaxel, adriamycin, tamoxifen, doxorubicin and mixtures thereof.

7. A method for alleviating symptoms, treating or preventing cancer, the method comprising administering to a subject, having or at risk of developing cancer, an effective amount of a pharmaceutical formulation comprising:
(a) one or more anticancer agents; and
(b) one or more phosphate derivatives of one or more hydroxy chromans selected from the group consisting of 7:8 dimethyl 6 hydroxy chromans, 8 methyl 6 hydroxy chromans and mixtures thereof.

8. The method according to claim 7 wherein the anticancer agent is lycopene or tamoxifen.

9. A method for increasing the efficacy of lycopene, the method comprising combining lycopene with one or more phosphate derivatives of one or more hydroxy chromans selected from the group consisting of 7:8 dimethyl 6 hydroxy chromans, 8 methyl 6 hydroxy chromans and mixtures thereof.

10. A method for increasing the efficacy of an anticancer agent, the method comprising combining the anticancer agent with one or more phosphate derivatives of one or more hydroxy chromans selected from the group consisting of 7:8 dimethyl 6 hydroxy chromans, 8 methyl 6 hydroxy chromans and mixtures thereof.

11. A pharmaceutical formulation comprising an effective amount of lycopene and an effective amount of one or more phosphate derivatives of one or more hydroxy chromans selected from the group consisting of 7:8 dimethyl 6 hydroxy chromans, 8 methyl 6 hydroxy chromans and mixtures thereof.

12. Use of an effective amount of one or more phosphate derivatives of one or more hydroxy chromans selected from the group consisting of 7:8 dimethyl 6 hydroxy chromans, 8 methyl 6 hydroxy chromans and mixtures thereof, together with a suitable carrier or diluent in the manufacture of a medicament for alleviating symptoms, treating or preventing cancer.

13. Use of one or more anticancer agents and one or more phosphate derivatives of one or more hydroxy chromans selected from the group consisting of 7:8 dimethyl 6 hydroxy chromans, 8 methyl 6 hydroxy chromans and mixtures thereof, together with a suitable carrier or diluent in the manufacture of a medicament for alleviating symptoms, treating or preventing cancer.

14. A pharmaceutical composition when used for inducing cell apoptosis, the composition comprising an effective amount of one or more phosphate derivatives of one or more hydroxy chromans selected from the group consisting of 7:8 dimethyl 6 hydroxy chromans, 8 methyl 6 hydroxy chromans and mixtures thereof.

15. A pharmaceutical formulation when used for alleviating symptoms, treating or preventing cancer, the formulation comprising one or more anticancer agents and one or more phosphate derivatives of one or more hydroxy chromans selected from the group consisting of 7:8 dimethyl 6 hydroxy chromans, 8 methyl 6 hydroxy chromans and mixtures thereof.

16. A method for inducing cell apoptosis comprising administering to cells an effective amount of one or more phosphate derivatives of one or more hydroxy chromans selected from the group consisting of 7:8 dimethyl 6 hydroxy chromans, 8 methyl 6 hydroxy chromans and mixtures thereof.

17. A method for inducing cell apoptosis comprising administering to cells an effective amount of a formulation comprising one or more anticancer agents and one or more phosphate derivatives of one or more hydroxy chromans selected from the group consisting of 7:8 dimethyl 6 hydroxy chromans, 8 methyl 6 hydroxy chromans and mixtures thereof.
